(19) United States
(12) Patent Application Publication
(10) Pub. No.: US 2020/0106736 A1
(43) Pub. Date: Apr. 2, 2020

(54) INFORMATION PROCESSING DEVICE AND INFORMATION PROCESSING METHOD

(71) Applicants: OMRON HEALTHCARE CO., LTD., Muko-shi (JP); OMRON Corporation, Kyoto-shi (JP)

(72) Inventors: Fumihiko NAKAMURA, Kyoto (JP); Daisuke NOZAKI, Kyoto (JP); Yasushi MATSUOKA, Kyoto (JP)

(21) Appl. No.: 16/699,906

(22) Filed: Dec. 2, 2019

Related U.S. Application Data
(63) Continuation of application No. PCT/JP2018/022222, filed on Jun. 11, 2018.

(30) Foreign Application Priority Data
Jun. 19, 2017 (JP) 2017-120014

(51) Int. Cl. H04L 12/58; G16H 50/30; G16H 10/60

(52) U.S. Cl. CPC H04L 51/38; G16H 10/60; G16H 50/30

(57) ABSTRACT

According to one embodiment, an information processing device includes an acquisition unit configured to acquire information regarding health of a plurality of subjects, a grouping process unit configured to form one or more groups including one or more subjects from among the plurality of subjects in accordance with the information regarding the health, and a creation unit configured to create a room in which to share posted messages in each of the one or more groups.

### CROSS-REFERENCE TO RELATED APPLICATIONS

This application is a Continuation Application of PCT Application No. PCT/JP2018/0222222, filed Jun. 11, 2018 and based upon and claiming the benefit of priority from Japanese Patent Application No. 2017-120014, filed Jun. 19, 2017, the entire contents of all of which are incorporated herein by reference.

### FIELD

Embodiments described herein relate generally to an information processing device, an information processing method, and an information processing program for processing information pertaining to the health of a subject.

### BACKGROUND

With the recent increase in health consciousness, increasing numbers of people are seeking health-care management advice from health-care workers. Health-care workers are required to provide an individual subject of health-care management with advice based on the understanding of the health issues of the health management subject, such as high blood pressure and suspected diseases.

Techniques for detecting the subject's health issues have been proposed. Jpn. Pat. Appln. KOKAI Publication No. 2013-233256 discloses a technique of employing heart rate fluctuation indices for diagnosis of mental illness and detection of an onset risk.

Health-care workers are required to offer health-care advice to all the subjects they are taking care of, in a face-to-face or electronic (such as chats) format. The task of offering advice to all the subjects, however, places a heavy burden on the health-care workers. Patent Literature 1 does not disclose the technique of reducing such a burden on the advice-giving health-care workers.

### SUMMARY

According to the first aspect of the present invention, an information processing device includes an acquisition unit configured to acquire information regarding health of a plurality of subjects, a grouping process unit configured to form one or more groups including one or more subjects from among the plurality of subjects in accordance with the information regarding the health, and a creation unit configured to create a room in which to share posted messages in each of the one or more groups.

A health-care worker can provide advice for each group, instead of each subject, which reduces the load of advice provision according to the first aspect of the present invention. The health-care worker can therefore improve the efficiency of tasks related to the health management of the subjects. In addition, the subject can use a chat room as a place for learning or communicating with other subjects who have similar concerns and problems.

According to the second aspect of the present invention, the information regarding health is acquired based on biological information of each of the subjects in the information processing device of the first aspect.

The information processing device according to the second aspect of the present invention can acquire highly accurate information on the health of the subject. In addition, the subject can omit the task of inputting health-related information.

According to the third aspect of the present invention, in the information processing device of the first aspect or the second aspect, each of the one or more groups includes one or more health-care workers.

The subject can use the room as a place for learning or communicating not only with subjects having similar concerns or problems but also with health-care workers according to the third aspect of the present invention.

According to the fourth aspect of the present invention, an information processing method includes a step of acquiring information regarding health of a plurality of subjects, a step of performing a grouping process to form one or more groups including one or more subjects from among the plurality of subjects in accordance with the information regarding their health, and a step of creating a room in which to share posted messages in each of the one or more groups.

The information processing method according to the fourth aspect of the present invention can attain effects similar to those of the first aspect described above.

According to the fifth aspect of the present invention, an information processing program is configured to cause a computer to function as the units of the information processing device according to any one of the first to third aspect.

The information processing program according to the fifth aspect of the present invention can attain effects similar to those of the first aspect described above.

The present invention offers a technique that aims at the improvement of efficiency in health-care workers' tasks related to the health management.

### BRIEF DESCRIPTION OF THE DRAWINGS

FIG. 1 is a diagram schematically illustrating an exemplary scene of the application of a server according to an embodiment.

FIG. 2 is a block diagram schematically illustrating an exemplary hardware configuration of the server according to the embodiment.

FIG. 3 is a block diagram schematically illustrating an exemplary configuration implemented by the software of the server according to the embodiment.

FIG. 4 is a diagram showing exemplary groups formed by the server according to the embodiment.

FIG. 5 is a diagram schematically illustrating an exemplary display screen of the mobile terminal according to the embodiment.

FIG. 6 is a flowchart illustrating an exemplary processing procedure of the server according to the embodiment.

### DETAILED DESCRIPTION

Embodiments according to the present invention will be described below with reference to the drawings.

An object to be accomplished by the embodiments is to provide an information processing device, an information processing method, and an information processing program, which can improve the efficiency of health management-related tasks of health-care workers.

### Application Examples

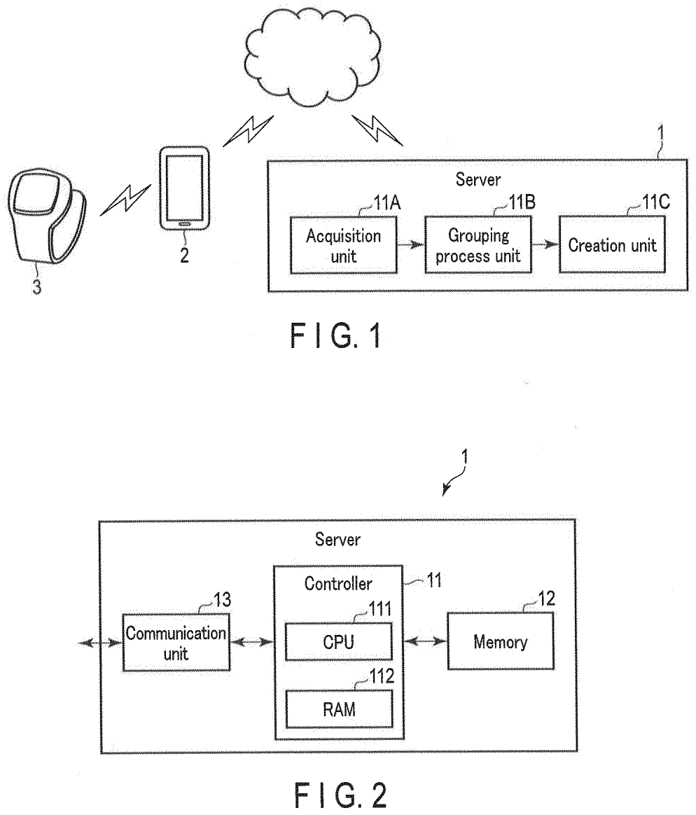

FIG. 1 is a diagram schematically illustrating an example of the application of a server according to an embodiment.

The server 1 includes an acquisition unit 11A, a grouping process unit 11B, and a creation unit 11C.

The acquisition unit 11A acquires information regarding the health of multiple subjects. In one example, the acquisition unit 11A may acquire information regarding the health of individual subjects based on information regarding one's own health previously input by each of the subjects. In another example, the acquisition unit 11A may acquire the biological information of individual subjects. The acquisition unit 11A may acquire information regarding the health of individual subjects based on the biological information of the individual subjects.

The grouping process unit 11B forms one or more groups including one or more subjects among the multiple subjects in accordance with the health information. Furthermore, the grouping process unit 11B may perform processing so that one or more groups each include one or more health-care workers.

The creation unit 11C creates a room in which to share the posted messages for each group. The members (one or more subjects and one or more health-care workers) of the group associated with the room are allowed to share messages posted by the members who belong to the group.

As described above, according to this embodiment, the efficiency in health management-related work by health-care workers can be improved.

### Structural Examples

### (Server)

### <Hardware Configuration>

FIG. 2 is a block diagram schematically illustrating an exemplary hardware configuration of the server 1 according to the embodiment. The server 1 is an example of an information processing device.

The server 1 includes a controller 11, a memory 12, and a communication unit 13.

The controller 11 controls the structural components of the server 1. The controller 11 includes a central processing unit (CPU) 111 and a random access memory (RAM) 112. The CPU 111 is an example of a processor. The controller 11 may include a plurality of processors. The controller 11 expands on the RAM 112 the program stored in the memory 12. The controller 11 interprets and executes the program expanded on the RAM 112 by way of the CPU 111, and controls each structural component of the server 1. The configuration of each component implemented in the controller 11 by the program will be described later.

The memory 12 stores programs and data. For example, the memory 12 is a non-volatile memory such as a hard disk drive or a solid state drive. The memory 12 stores programs that cause the controller 11 or the CPU 111 to function as the components included in the server 1. The program may also be referred to as a command for operating the controller 11 or the CPU 111. The memory 12 stores data used for controlling the server 1, data used for setting various functions of the server 1, and the like.

The communication unit 13 is an interface for wirelessly communicating with a device which differs from the server 1. The communication unit 13 includes a wireless local area network (LAN) module. The communication unit 13 communicates with a mobile terminal 2 via a network. The communication unit 13 outputs to the controller 11 information received from the mobile terminal 2 via the network. The network may be, but is not limited to, the Internet.

The mobile terminal 2 will be described. The mobile terminal 2 may be, but is not limited to, a smartphone or a tablet terminal. The mobile terminal 2 can acquire blood pressure values (systolic blood pressure (SBP) and diastolic blood pressure (DBP)) from the blood pressure monitor 3 through short-range wireless communications such as Bluetooth (registered trademark). The mobile terminal 2 sends the blood pressure values to the server 1. The blood pressure monitor 3 is an example of a biological information measurement device. A blood pressure value is an example of the biological information. The mobile terminal 2 may acquire biological information from a type of biological information measurement device other than the blood pressure monitor 3, and transmit this biological information to the server 1. The biological information may include any elements related to the subject, such as an electrocardiographic pattern, pulse rate, body temperature, bone density, height, weight, body fat percentage, amount of activity and number of steps the subject takes, but is not limited to these.

### <Configuration Implemented by Software>

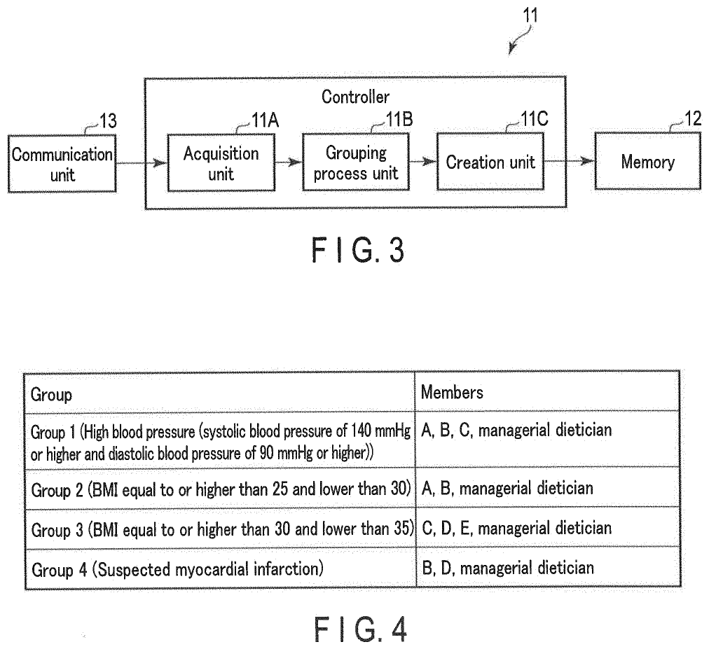

FIG. 3 is a block diagram schematically illustrating an exemplary configuration implemented by the program of the server 1 according to the embodiment.

The controller 11 includes an acquisition unit 11A, a grouping process unit 11B, and a creation unit 11C.

The acquisition unit 11A will be described.

The acquisition unit 11A acquires information regarding the health of multiple subjects.

In one example, the health information indicates any of one or more ranks defined as the health index. For example, the index may be, but is not limited to, a blood pressure value or Body Mass Index (BMI). When the index is a blood pressure value, a rank corresponding to a high blood pressure (systolic blood pressure being 140 mmHg or higher and diastolic blood pressure being 90 mmHg or higher), for example, may be determined. When the indicator is BMI, a rank corresponding to a BMI equal to or higher than 25 and lower than 30, and a rank corresponding to a BMI equal to or higher than 30 and lower than 35 may be determined. The number of ranks determined in accordance with the index and the numerical range corresponding to each rank can be freely set and changed.

In another example, the health information may indicate whether any disease is suspected. Such diseases may include, but are not limited to, myocardial infarction.

Next, an example of the acquisition unit 11A acquiring information related to the health of multiple subjects will be described.

In one example, the acquisition unit 11A may acquire information regarding the health of individual subjects based on information regarding one's own health previously input by each of the subjects. Here, the acquisition unit 11A acquiring the information regarding the health of a given subject will be described as an example. For example, the acquisition unit 11A operates in cooperation with the mobile terminal 2 to acquire information regarding the health of the subject, as described below.

The subject activates a specific application on the mobile terminal 2 and inputs information regarding his/her own health. For example, if the subject's blood pressure values fall into the rank corresponding to high blood pressure, this rank corresponding to high blood pressure is input as an index of blood pressure value. Similarly, if the subject's BMI falls into any one of the one or more ranks defined in relation to the BMI index, this rank is input. The subject also inputs whether or not any disease is suspected for him/herself. The mobile terminal 2 transmits the health information based on the input by the subject as well as the subject's identification information, to the server 1.

The acquisition unit 11A acquires the subject's health information and identification information from the mobile terminal 2 via the communication unit 13. The acquisition unit 11A enters the subject's health information and identification information into the memory 12. The memory 12 stores the subject's health information in association with this subject's identification information.

Similarly, the acquisition unit 11A can acquire information regarding the health of multiple subjects. In this manner, the memory 12 can store health information and identification information in association with each subject.

In another example, the acquisition unit 11A may acquire information regarding the health of individual subjects from the biological information of the individual subjects collected by the mobile terminal 2, in place of each of the subjects inputting the information regarding his/her own health. Here, the acquisition unit 11A acquiring information regarding the health of a given subject will be described as an example. The acquisition unit 11A may operate in cooperation with the mobile terminal 2 to acquire the subject's health information, as described below.

The mobile terminal 2 collects the biological information of the subject. In one example, the mobile terminal 2 may communicate with the biological information measurement device to collect the subject's biological information measured by the biological information measurement device. For example, the mobile terminal 2 may communicate with the blood pressure monitor 3 to collect the blood pressure values of the subject measured by the blood pressure monitor 3. The mobile terminal 2 may communicate with the weight scale to collect the weight of the subject measured by the weight scale. In another example, the mobile terminal 2 may collect the biological information of the subject, based on the biological information that is input by the subject. For example, the subject may activate a specific application on the mobile terminal 2 to input his/her own blood pressure values or body weight. In this manner, the mobile terminal 2 can collect the subject's blood pressure values or body weight. The mobile terminal 2 transmits to the server 1 the subject's biological information and the subject's identification information associated with the biological information.

The acquisition unit 11A acquires, from the mobile terminal 2 via the communication unit 13, the subject's biological information and identification information collected by the mobile terminal 2. The acquisition unit 11A enters into the memory 12 the subject's biological information and identification information. The memory 12 stores the subject's biological information in association with the subject's identification information. Similarly, the acquisition unit 11A can acquire biological information of multiple subjects. In this manner, the memory 12 can store the biological information and identification information in association with each subject.

The acquisition unit 11A acquires the subject's health information based on the biological information acquired as described above.

The acquisition unit 11A may acquire, as the health information, the information indicating any of the one or more ranks defined with regard to the health index, based on the biological information as described below.

In one example, the acquisition unit 11A refers to the blood pressure values as biological information. In this case, the acquisition unit 11A determines whether or not the subject's blood pressure values fall into a rank corresponding to high blood pressure. If the subject's blood pressure values fall into the rank corresponding to high blood pressure, the acquisition unit 11A acquires the information indicating the rank corresponding to high blood pressure, as the subject's health information.

In another example, the acquisition unit 11A refers to the height and weight of the subject as biological information. In this case, the acquisition unit 11A calculates the BMI of the subject based on the height and weight of the subject. The acquisition unit 11A determines whether or not the subject's BMI falls into any of the one or more ranks defined with regard to the BMI index. If the subject's BMI falls into any of the ranks, the acquisition unit 11A acquires the information indicating the rank into which the subject's BMI falls, as the subject's health information.

The acquisition unit 11A may acquire information indicating whether a disease is suspected, as the health information based on the biological information as described below.

The acquisition unit 11A may refer to guidelines that define the relationship between the biological information and suspected diseases. For example, the guidelines may define a case in which an element X1 of the biological information does not satisfy a reference value Y1, as a suspected disease Z1. The guidelines may further define a case in which an element X2 of the biological information does not satisfy a reference value Y2 and an element X3 does not satisfy a reference value Y3, as a suspected disease Z2. In this manner, the acquisition unit 11A can acquire information regarding presence or absence of a suspected disease based on the biological information. The acquisition unit 11A enters the subject's health information into the memory 12. The memory 12 stores the subject's health information in association with this subject's identification information. Similarly, the acquisition unit 11A can acquire information regarding the health of multiple subjects. In this manner, the memory 12 can store health information and identification information in association with each subject.

The grouping process unit 11B will be described.

The grouping process unit 11B may form one or more groups including one or more subjects from among the multiple subjects in accordance with the health information, as described below.

The grouping process unit 11B refers to the health information stored in the memory 12 in association with individual subjects. The grouping process unit 11B extracts all the subjects who are associated with the first set of health-related information, from all the subjects stored in the memory 12. The grouping process unit 11B forms a group that includes all the subjects associated with the first set of health-related information. All the subjects associated with the first set of health-related information belong to the group corresponding to the first set of health-related information. Similarly, the grouping process unit 11B extracts all the subjects associated with the second set of health-related information, from all the subjects stored in the memory 12. The grouping process unit 11B forms a group including all the subjects associated with the second set of health-related information. All the subjects associated with the second set of health-related information belong to the group corresponding to the second set of health-related information.

Some examples of the groups formed by the grouping process unit 11B will be described later.

The grouping process unit 11B may further perform processing so that one or more groups as formed in the above manner each include one or more health-care workers. A health-care worker is a person who has knowledge regarding health. The health-care worker may be, but is not limited to, a public health nurse or managerial dietician.

The grouping process unit 11B enters the information regarding one or more groups into the memory 12. The grouping process unit 11B outputs the information regarding the one or more groups to the creation unit 11C.

The creation unit 11C will be described.

The creation unit 11C creates a room in which to share posted messages in each of one or more groups, as described below.

The creation unit 11C acquires information regarding one or more groups from the grouping process unit 11B. The creation unit 11C refers to the information related to the one or more groups, and creates a room in which to share posted messages in each of the one or more groups. In a room, the members (one or more subjects and one or more health-care workers) of the group associated with this room can share messages posted by the members who belong to this group. The creation unit 11C stores the rooms corresponding to individual groups in the memory 12.

An exemplary display screen on the mobile terminal 2 of the room created by the creation unit 11C will be described later.

### (Example of Group Structure)

FIG. 4 is a diagram showing exemplary groups formed by the server 1 according to the embodiment.

The grouping process unit 11B may form Groups 1 to 4, for example, including one or more subjects from among the multiple subjects, in accordance with the health information.

The grouping process unit 11B forms Group 1, which includes one or more subjects and managerial dieticians and is associated with the information indicating a rank corresponding to high blood pressure. The grouping process unit 11B forms Group 2, which includes one or more subjects and managerial dieticians and is associated with information indicating a rank corresponding to a BMI equal to or higher than 25 and lower than 30. The grouping process unit 11B forms Group 3 including one or more subjects and managerial dieticians and associated with information indicating a rank corresponding to a BMI equal to or higher than 30 and lower than 35. The grouping process unit 11B forms Group 4 including one or more subjects and managerial dieticians and associated with information indicating a suspected myocardial infarction.

Group 1 is a group corresponding to health information which differs from Group 2, 3 or 4. This means that the members of Group 1 may overlap with the members of Group 2 or 3. Similarly, the members of Group 1 may overlap with the members of Group 4.

Groups 2 and 3 are both based on an index of BMI. For this reason, the members of Group 3 would not overlap with the members of Group 2, except for the managerial dieticians.

### (Exemplary Display in Room)

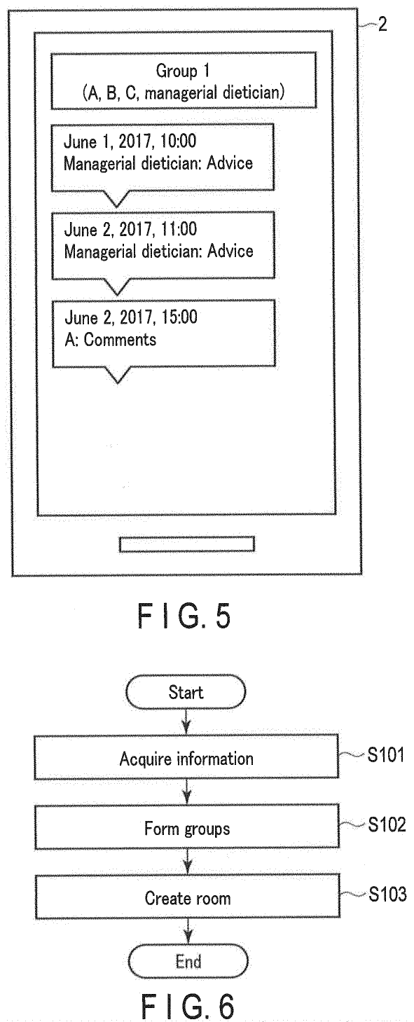

FIG. 5 is a diagram schematically illustrating an exemplary display screen of the mobile terminal 2 according to the embodiment.

It is assumed here that the owner of the mobile terminal 2 is the subject A. The mobile terminal 2 can download the rooms of one or more groups to which the subject A belongs, from the server 1. The room is to share posted messages within each of one or more groups. The mobile terminal 2 can display the room of one group selected from the one or more groups to which the subject A belongs. As shown in FIG. 5, the mobile terminal 2 can display the room of Group 1 as shown in FIG. 5, based on the instructions for displaying the room of Group 1 from the subject A.

The mobile terminal 2 sends to the server 1 a message to be posted, based on the input of the message to be posted in the room of Group 1 by the subject A. At the same time, the mobile terminal 2 displays the posted message in the room of Group 1 that has been downloaded onto the mobile terminal 2. The mobile terminal 2 receives messages posted by the members of Group 1 other than the subject A from the server 1, and displays them in the room of Group 1 downloaded onto the mobile terminal 2.

In this manner, only the members who belong to Group 1 (one or more subjects and one or more health-care workers) can upload messages to be posted in the room of Group 1. The posted messages uploaded in the room of Group 1 cannot be viewed by a person who does not belong to Group 1.

### Operational Examples

### (Operation of Server)

FIG. 6 is a flowchart illustrating an exemplary processing procedure of the server 1 according to the embodiment. The processing procedure is described below merely as an example, and changes may be made to the operations as appropriate. In addition, the processing procedure described below can involve omission, replacement, and addition of steps as appropriate.

The acquisition unit 11A acquires information regarding the health of multiple subjects (step S101). At step S101, the acquisition unit 11A can acquire the health information for individual subjects, as shown in the example.

The grouping process unit 11B forms one or more groups including one or more subjects from among the multiple subjects in accordance with the health information (step S102). At step S102, the grouping process unit 11B can form one or more groups, as in the above described examples. At step S102, the grouping process unit 11B may also perform processing so that each of one or more groups includes one or more health-care workers.

The creation unit 11C creates a room in which to share the posted messages within each of the one or more groups (step S103). At step S103, the creation unit 11C can create one or more rooms, as in the above described examples.

Even after creating the room, the server 1 can add one or more new subjects to the group associated with this room.

### Operations and Effects

As detailed above, the server 1 according to the embodiment of the present invention forms one or more groups including one or more subjects from among the multiple subjects in accordance with the health information, and creates a room to share the posted messages within each of the groups. In this manner, the health-care workers can offer advice for each group, instead of each subject, which can reduce the burden of the task of offering advice. As a result, the efficiency of the health-care worker's tasks related to the health management of the subjects can be enhanced. In addition, the room can be used by the subject as a place for learning or communicating with other subjects who have similar concerns and problems.

According to the embodiment of the present invention, the server 1 can acquire information regarding the health of individual subjects, based on the biological information of the individual subjects. Thus, the server 1 can obtain highly accurate information regarding the health of the subjects. In addition, the subject can now omit the task of inputting the health information.

In the embodiment of the present invention, the server 1 can process each of one or more groups so as to include one or more health-care workers. As a result, the subject can use the room as a place for learning or communicating not only with subjects having similar concerns or problems but also with health-care workers.

### Modification Examples

In addition, the above embodiment may be modified as indicated below. In the following, the same reference numerals are used for the structural components similar to those in the above embodiment, and the descriptions of the same points as in the above embodiment are omitted as appropriate. The following modifications can be combined as appropriate.

In other words, the present invention is not limited to the above-described embodiment as it is, and can be embodied by modifying the structural component without departing from the scope of the invention in the implementation stage. In addition, various other inventions can be configured by appropriately combining structural components disclosed in the embodiment. For example, some of the structural components shown in the embodiment may be omitted. Furthermore, the structural components of different embodiments may be appropriately combined.

At least part of the processing of the above embodiment can be realized by using a general-purpose computer as basic hardware. The program for realizing the above processing may be stored and provided in a computer-readable storage medium. The program is stored in the storage medium as a file of either installable or executable format. The storage medium will suffice as long as it can store programs and can be read by the computer. A storage media includes magnetic disks, optical disks (compact disk-read only memory (CD-ROM), compact disk-recordable (CD-R), digital versatile disk (DVD), etc.), magneto-optical disks (MO), and a semiconductor memory. The program for realizing the above processing may be stored on a computer (server) connected to a network such as the Internet and downloaded to the computer (client) via the network.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An information processing device comprising a processor and a memory, wherein the processor is configured to:
acquire information that includes at least one of indices of different types related to health of a plurality of subjects who are targeted for health management;
form a plurality of first groups corresponding to the indices of different types related to the health and a plurality of second groups corresponding to an index of a same type and having different numerical ranges such that, based on the acquired health-related information, the subjects targeted for the health management are placed into a corresponding one or more of the first groups in an overlapping manner among the groups, and also placed into a corresponding one of the second groups in a non-overlapping manner among the groups, wherein one or more health-care workers belong to each of the first groups and the second groups; and
create a room in which to share posted messages in each of the first groups and the second groups.

2. The device according to claim 1, wherein the information regarding the health is acquired based on biological information of each of the subjects.

3. A non-transitory computer readable medium storing a computer program which is executed by a computer to provide the steps of:
acquiring information that includes at least one of indices of different types related to health of a plurality of subjects who are targeted for health management;
forming a plurality of first groups corresponding to the indices of different types related to the health and a plurality of second groups corresponding to an index of a same type and having different numerical ranges such that, based on the acquired health-related information, the individual subjects targeted for the health management are placed into a corresponding one or more of the first groups in an overlapping manner among the groups, and also placed into a corresponding one of the second groups in a non-overlapping manner among the groups, wherein one or more health-care workers belong to each of the first groups and the second groups; and
creating a room in which to share posted messages in each of the first groups and the second groups.

4. An information processing method configured to be implemented by an information processing device comprising a processor and a memory, comprising:
acquiring, by the information processing device, information that includes at least one of indices of different types related to health of a plurality of subjects who are targeted for health management;
forming, by the information processing device, a plurality of first groups corresponding to the indices of different types related to the health and a plurality of second groups corresponding to an index of a same type and having different numerical ranges such that, based on the acquired health-related information, the individual subjects targeted for the health management are placed into a corresponding one or more of the first groups in an overlapping manner among the groups, and also placed into a corresponding one of the second groups in a non-overlapping manner among the groups, wherein one or more health-care workers belong to each of the first groups and the second groups; and
creating, by the information processing device, a room in which to share posted messages in each of the first groups and the second groups.
